(19) United States
(12) Patent Application Publication
(10) Pub. No.: US 2011/0295300 A1
(43) Pub. Date: Dec. 1, 2011

(54) INTRAGASTRIC TREATMENT ASSEMBLY

(76) Inventors: Jean-Michel Verd, Saint Chamond (FR); Pierre-André Denis, Villeurbanne (FR)

(21) Appl. No.: 13/147,531

(22) PCT Filed: Nov. 19, 2009
(86) PCT No.: PCT/FR09/52227

§ 371 Date: Aug. 2, 2011

(30) Foreign Application Priority Data
Feb. 4, 2009 (FR) 0950689

(51) Int. Cl. A61M 29/00

(52) U.S. Cl. 606/192

(57) ABSTRACT

This invention relates to an assembly comprising an intragastric balloon (101) and inflation needle (125), said balloon comprising a flexible pouch (102) that is airtight and impermeable to food liquids or physiological fluids, as well as an inflating valve (111) made of elastomer, one end of which (112) is accessible from the outside of the balloon while the opposite end the opposite end (113) is located inside the balloon, and comprises: a channel (117), open at the exterior end, for receiving an inflation needle, an inflation channel (118) open at the interior end and coaxial with the reception channel, and a septum (119) separating the reception channel from the inflation channel. The reception channel and the inflation needle are adapted, by relative configuration, in order that, when the inflation needle is inserted into the reception channel without being inserted in the septum (19; 119), the inflation needle is held in place in the reception channel.

The present invention relates to the technical field of intragastric balloons, used particularly in the treatment of obesity.

An intragastric balloon is generally shaped like a flexible pouch that is airtight and made impermeable to food liquids or physiological fluids. The flexible pouch is also equipped with an inflating valve designed to enable an inflating fluid, such as air or a physiological saline solution, to be injected once the balloon has been fitted inside the patient's stomach. One patent, U.S. Pat. No. 5,084,061, describes, for example, a balloon comprising an inflating valve formed by a channel sealed by a series of two split diaphragms and opening out from a tubular silicone cone acting as a valve. Intragastric balloons comprising inflatable valves such as these have the disadvantage of being difficult to inflate, in light of the complexity of the operation of inserting an inflating device into a valve while the balloon is located inside the patient's stomach.

In order to remedy this disadvantage, a proposal has been made, for example in U.S. Pat. No. 4,723,547, in favour of intragastric balloons which are pre-fitted with an inflating device with the needle fully inserted into the inflating valve, in such a way as to be in flow communication with the inside of the pouch of the balloon. The needle is then inserted into the valve well before the balloon is fitted inside the patient's stomach, more specifically, when the assembly of the inflation device and the balloon has been completed, before the balloon is delivered to its operating location. Such balloons were found to exhibit problems with regard to impermeability during use, which caused them to spontaneously deflate after being placed in the patient's stomach.

It is therefore considered necessary to have a new type of balloon, which is very easy to inflate once inserted into the patient's stomach while at the same time having optimum impermeability so as to minimise the risks of spontaneous deflation of the balloon, except in the event of perforation of the flexible pouch of the balloon.

To this end, the invention is intended to be an intragastric treatment assembly, as defined in claim 1.

The implementation of a channel for receiving an inflation needle, very appropriately enables the inflation needle to be positioned in advance before the balloon is inserted into the patient's stomach. In view of the relative dimensions of the reception channel and the needle, the needle is reliably and securely fixed in place when the pre-positioning configuration is used. In other words, based on the invention, the reception channel and the needle fit together and are therefore adapted for ensuring that the needle is held in place before it is inserted into the septum. As the needle is pre-positioned without being inserted into the septum, no deterioration of the septum is likely to occur. The intragastric balloon can be kept in this state for long periods without the risk of the septum “memorising” the shape of the needle and therefore having an effect on its impermeability. Moreover, to the extent that the septum is composed of a thick material which is not penetrated by the needle while the balloon is in storage before use, it has excellent impermeability characteristics as being self-sealant. In addition, the implementation of an inflation channel prevents any risk of the flexible pouch of the balloon being perforated by the inflation needle.

For all practical purposes, therefore, the inflation needle is not inserted into the septum of the inflating valve while the balloon is in storage so as to preserve its integrity prior to its implementation in the balloon. The needle is therefore only inserted into the septum immediately prior to being fitted inside the patient's stomach.

Further beneficial characteristics are specified in claims 2 and 3.

Appropriately, the inner diameter of the inflation channel can be greater than the outer diameter of the needle. Consequently, the implementation of an enlarged inflation channel in relation to the diameter of the needle prevents the inflation channel from resisting the insertion of the needle.

According to the invention, the elongate core is likely to be composed of various types of elastomers, preferably synthetic, in order to minimise the risk of patient allergies. As a result, the elongate core may be composed of biocompatible silicone.

In order to facilitate sealable attachment of the valve to the flexible pouch, the inflating valve includes a main body made of a polymer material, along the inside of which extends the elongate core which is secured to the pouch of the balloon. The body of the valve can thus be composed of the same kind material as the constituent material of the flexible pouch or even of a material having optimum compatibility with the pouch in order for it to be assembled by means of welding or cementing.

According to another feature of the invention, the valve can include a prestressing ring encircling at least a part of the area of the core forming the septum. Such a prestressing ring can thus encircle only the elongate core or the assembly formed by the main body and the elongate core. The implementation of such a ring, by means of centripetal pressure applied to the core, increases the capacity of the part of the core forming the septum to close after the inflation needle is withdrawn, which corresponds to the septum's auto-sealing feature.

According to one variant in the implementation of this feature, the prestressing ring is composed of radio-opaque inflexible material. The use of such a material, as its name indicates, facilitates tracking of the intragastric balloon in a patient's digestive system by means of x-ray radiography. Thus, the prestressing ring can, for example, be composed of metal, preferably but not necessarily nonmagnetic, so as to avoid interactions with medical magnetic resonance imaging devices. The prestressing ring can also be composed of thermoplastic material loaded with radio-opaque material, such as BaSO4.

According to the invention, the flexible pouch of the intragastric balloon is likely to be composed of any appropriate material having suitable characteristics of air-tightness and imperviousness to food liquids or physiological liquids, as well as resistance to the acidity of gastric juices. The flexible pouch can also, for example but not necessarily, be composed of one or more single-layer polyurethane sheets between 0.1 mm and 0.4 mm thick. Consequently, the flexible pouch can be composed by assembling, in juxtaposition, sheets of flexible material of various shapes, such as essentially triangular or lentoid. According to the invention, the pouch can also be composed of two discs, identical in diameter, assembled edge to edge. The assembly of the pre-cut sheets constituting the core can also be achieved using any appropriate method or procedure, such as, for example, cementing or high-frequency welding.

In order to facilitate the work of the physician who will be fitting the intragastric pouch, preferably through natural passageways, the balloon can be combined with methods facilitating and/or controlling this installation. Various corresponding forms of embodiment are specified in claims 11 to 17.

Naturally, the various features, variants and forms of embodiment of the intragastric treatment assembly according to the invention can be implemented with another according to various combinations, inasmuch as they are not incompatible with each other or mutually exclusive.

In addition, various other features and advantages of the invention will be revealed from the description of the attached diagrams illustrating various non-restrictive forms of embodiment:

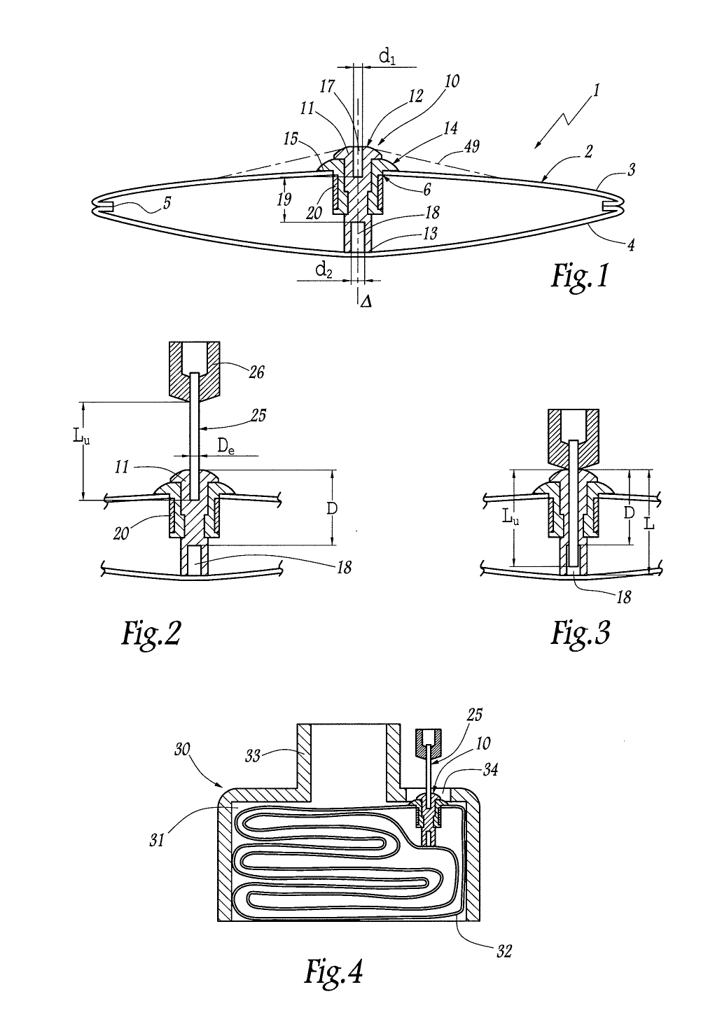

FIG. 1 is a diagrammatic cross-section of an intragastric balloon belonging to an assembly according to the invention;

FIGS. 2 and 3 are larger scale longitudinal cross-sections of the inflating valve of the balloon illustrated in FIG. 1 and show two stages of the embodiment of the inflating valve with an inflation needle belonging to the assembly according to the invention;

FIG. 4 is a diagrammatic cross-section of an intragastric balloon as illustrated in FIG. 1, folded inside an endoscope cap belonging to an assembly according to the invention;

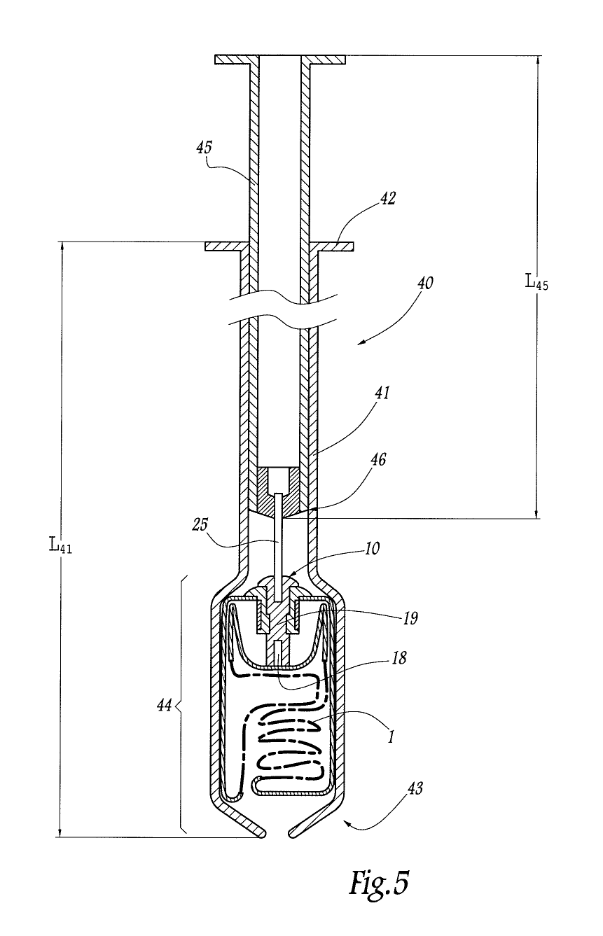

FIG. 5 is a diagrammatic longitudinal cross-section of another form of embodiment of an assembly according to the invention;

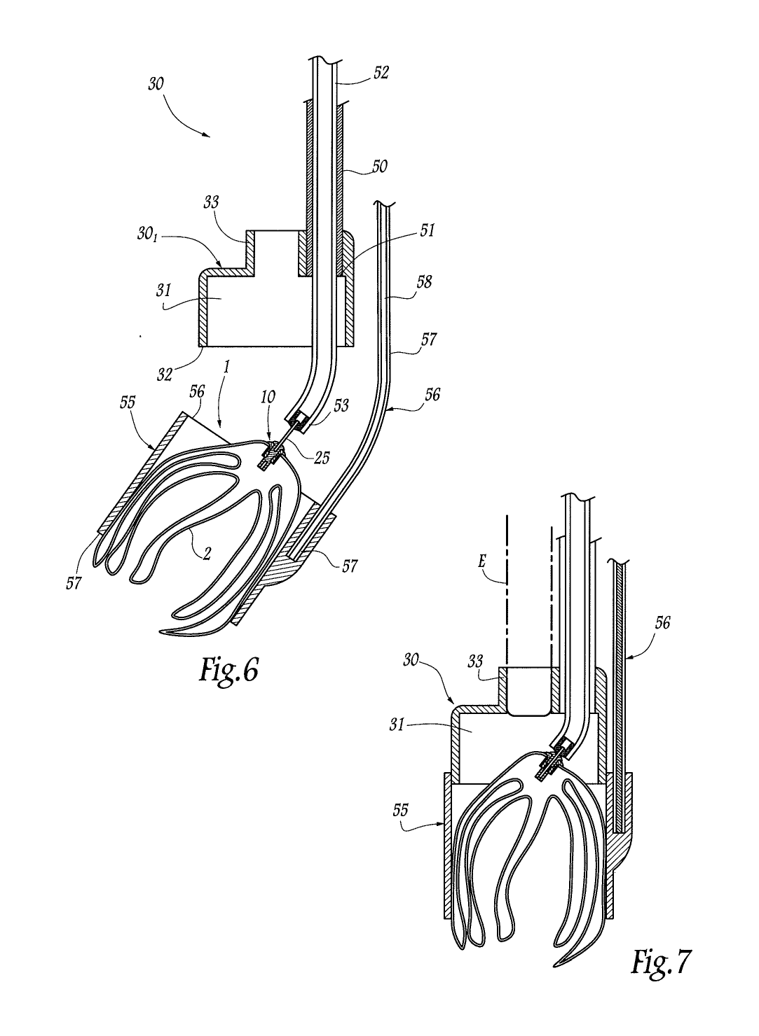

FIGS. 6 and 7 are diagrammatic longitudinal cross-sections of two utilisation phases, of another form of embodiment of an assembly according to the invention; and

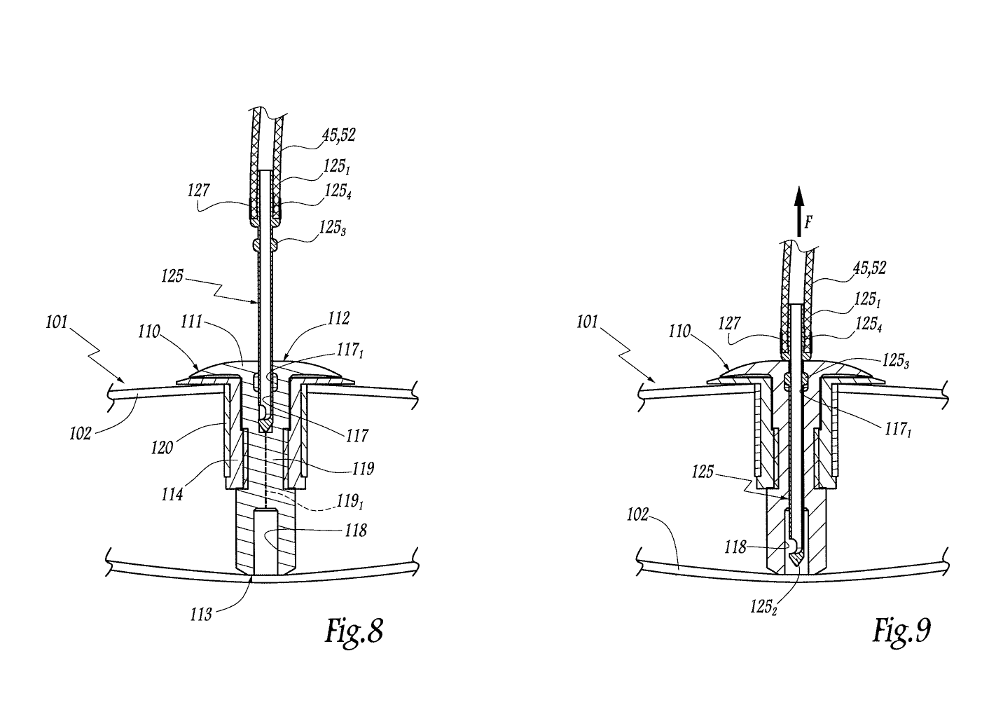

FIGS. 8 and 9 are views similar to FIGS. 2 and 3, respectively, illustrating a variant of embodiment in keeping with the invention.

### It should be noted that the elements common to the various forms of embodiment have the same references in the figures.

An intragastric balloon conforming to the invention, as illustrated in FIG. 1, and designated in its assembly by reference 1, includes a flexible pouch 2 composed of an airtight material impervious to food liquids or physiological fluids. According to the example shown, the pouch 2 is formed by the assembly of two single-layer polyurethane disks 3 and 4, with a thickness e between 0.1 mm and 0.4 mm and, for example but not exclusively, of approximately 200 μm. The two disks are assembled edge to edge by means of circumferential welding 5. The value of the external radius of the disks will be chosen so that the nominal volume of the inflated balloon 1 ranges preferably between 400 cm³ and 800 cm³. Allowance can be made, for example, for balloons having a nominal capacity of 500 cm³ and for others having a nominal capacity of 700 cm³, with the size of the balloon used being chosen based on the dimensions of the patient's stomach or on the obesity of the patient. For the construction of a balloon which will be inflated to have a nominal volume of 700 cm³, two disks 7 cm in diameter can be used whereas for constructing a balloon which will be inflated so that it has a nominal volume of 700 cm³, two disks 11 cm in diameter, for example, can be used.

The pouch 2 formed by assembly of the disks 3 and 4 is preferably pulled up through an opening 6 made in the upper disk 3 for the positioning of an inflating valve 10. By pulling up the pouch 2 this enables the circumferential weld seam to be located inside the balloon 1, so that the balloon provides an exterior surface area without protuberances, thus reducing the risk of irritation of the gastric wall after implantation.

According to the example shown, the inflating valve 10 includes an elongate core 11 composed of elastomer, and in the present case, of medical silicone. The core 11 therefore has one end 12 located outside the balloon and an opposite end 13 located inside the balloon. The core 11 is in addition surrounded by a main body 14 composed of a polymer material compatible with the material constituting the flexible pouch 2. According to the example shown, the main body 14 is composed of polyurethane and has at one end facing the exterior end 12 the core, a welded flange 15 on the upper disk 3. The main body 14 and the core 11 have complementary configurations for ensuring translation immobility of the core 11 in relation to the main body 14 while ensuring that their contact area is completely impermeable. It should be noted that according to the examples shown, the core 11 extends along a Δ axis having rotational symmetry in relation to this axis. The elongate core 11 additionally includes a channel 17 for receiving an inflation needle. The reception channel 17 is open at the exterior end 12 and is essentially in the general shape of a rotational cylinder with a Δ axis. The core 11 comprises at the opposite end of the reception channel 17, an inflation channel 18 which is open at the interior end 13. The inflation channel 18 is coaxial with the reception channel 17 and is in the general shape of a rotational cylinder with a Δ axis. The core 11 thus comprises, between the reception channel 17 and the inflation channel 18, a septum 19 which provides an airtight separation between the reception channel 17 and the inflation channel 18.

According to the example shown, the valve 10 also comprises a ring 20 encircling both the body 14 and the core 11 over at least one part of the septum 19, is such a way as to apply centripetal force to the septum in order to support its self-sealing feature, as will be subsequently shown.

The intragastric balloon 1 equipped with such an inflating valve 10 is intended for particular use in combination with an inflation needle 25, as illustrated in FIG. 2. This needle 25 can for example comprise an adaptor 26 on a catheter or an inflation tube (not shown). Naturally, the needle 25 can also be directly fitted to the end of an inflation tube. According to the example, the needle 25 is composed of a metal tube the free end of which will preferably be soft shaped so as to prevent the valve 10 from being damaged when the needle is inserted. In addition, the needle 25 has a usable length L_{U} and an external diameter Dₑ.

The core 11 therefore has a length L strictly greater than the usable length L_{U} of the needle 25, as shown in FIG. 3. In order to ensure that the needle 25 is properly held in place when inserted into the reception channel 17 only, this channel has an inner diameter d₁, smaller or equal to the external diameter D. of the needle. By preference, the inner diameter d₁ of the reception channel will be chosen so as to ensure a tight fit with the needle 25 and thus avoid accidental withdrawal of the needle when it is inserted just into the reception channel 17. In order to ensure the stability of this insertion, the reception channel 17 can be chosen so as to have a length greater than 1 mm, ranging for example between 1 mm an 8 mm. According to the example shown, the reception channel 17 is between 2 mm and 6 mm in length. In addition, in order to limit resistance from the valve when the needle 25 has completely passed through the septum, the inflation channel 18 will preferably have a lower diameter d₂ which is larger than the outer diameter Dₑ.

In order to allow flow communication between the inside of the needle and the inflation channel 18, the distance D separating the exterior end 12 of the core from the inner surface of the septum 19, located near the inflation channel 18 will be chosen so that it is shorter than the usable length L_{U} of the inflation needle. It should be noted that, in consideration of the value chosen for the length L of the core in relation to the usable length L_{U} of the needle, the end of the needle is located inside the inflation channel 18 even while the needle is completely embedded and thus prevents any risk of damage to the pouch 2 of the balloon by the free end of the needle 25. It should further be noted that when the needle is removed, the septum closes naturally, such self-sealing being favoured by the pressure applied by the ring 20 which, according to the example shown, can be made of stainless steel, preferably non-magnetic.

The intragastric balloon 1, composed as such, can be installed in various ways in a patient's stomach by a professional or an operator. The balloon can, for example, be lowered directly into the stomach through natural passageways, without any special accessories. The balloon can also be installed by means of an lead-in device formed by an endoscope cap 30 as illustrated in FIG. 4.

According to this example, the intragastric balloon 1 is folded on the inside of a chamber 31 of the endoscope cap 30. The chamber 31 is in the general shape of a cylinder and opens at a distal opening 32. The endoscope cap 30 comprises, in addition, a tubular collar 33 to be fitted onto an endoscope (not represented). The tubular collar 33 leads to the receiving chamber 31 opposite the distal opening 32. The intragastric balloon is then folded inside the chamber 31 so that the exterior end 12 of the inflating valve 10 is located next to an inflation opening 34 created on the upper part of the chamber 31 beside the adaptor collar 33. The inflation opening 34 provides access to the valve 10 and allows the inflation needle 25 to be pre-positioned in the reception channel 17.

Before implantation, the operator takes care to completely insert the needle in the valve 10, connects the needle to an inflation tube if this has not been done, then places the cap 30 on the end of an endoscope. The operator then inserts the assembly into the patient's stomach via the oesophagus. Once the cap and the end of the endoscope to which it has been fitted are inside the stomach, the operator proceeds to inflate the balloon, which will be instrumental in expelling it from the receiving chamber 31.

The balloon can be inflated using any suitable fluid such as, for example, a biocompatible gas like nitrogen or medical carbon dioxide or even air. The inflation fluid can also be a biocompatible liquid such as, for example, water, edible oil or physiological saline solution. Inflation can also be achieved using gas or liquid. In the latter case, the gas used can be air and physiological saline solution or an edible oil combined if necessary with a biocompatible colouring agent that can be eliminated by the kidneys, such as methylene blue. A colouring agent such as this makes perforations or accidental leaks easily detectable by means of staining the patient's urine.

In order to facilitate the operator's task, in addition to the intragastric balloon, he can be provided with one or more syringes pre-filled with the volume of liquid to be used. In order to give the intragastric balloon 1 in the patient's stomach a certain degree of stability, it can be filled with a volume of liquid representing between 5% and 20% of the nominal volume of the balloon. In the case of a 700 cm³ balloon, the operator can be provided with a syringe pre-filled with 70 cm₃ of a mixture of physiological saline solution and methylene blue. Other examples of how inflation is achieved are given at the end of this description.

Inflation of the balloon is achieved in the following manner. The operator first injects the contents of the pre-filled syringe into the balloon via the inflation tube. The operator then completes filling the balloon by successively injecting air and fluids using the same syringe or another syringe. In order to facilitate this operation, the inflation tube can be fitted with a three-way valve, with one valve connected to the inflation tube, another to a vent equipped if necessary with a non-return valve permitting suction and the final channel connected to the filling syringe. With each injection the operator controls the volume of air introduced into the balloon and injects as many injections as are necessary to enable the intragastric balloon 1 to achieve its nominal volume.

When inflation is completed, the operator removes the inflation tube and the endoscope, having visually checked via the endoscope that the balloon has been properly filled.

The intragastric balloon 1 according to the invention can also be installed by means of any other suitable lead-in device. Hence, FIG. 5 illustrates another form of lead-in device combined with the intragastric balloon according to the invention.

This oesophageal lead-in device designated in its assembly by the reference 40, comprises a lead-in tube 41 elongate in shape and flexible at least in part. The tube 41 has a proximal end 42, opposite which is a distal end 43. The length L₄₁ of the lead-in tube is therefore chosen so that when the distal end 43 is located inside a patient's stomach, the proximal end 42 is located on the exterior so as to enable it to be manoeuvred and held in place by the operator. The length L₄₁ will, for example, be chosen so that it ranges between 47 cm and 70 cm, by being, for example, in the region of 50 cm.

The lead-in tube 41 additionally has at its distal end 43 a tubular chamber 44 for receiving the intragastric balloon 1 in its folded state. According to the example illustrated, the receiving chamber 44 has an enlarged diameter compared to the rest of the lead-in tube 41. The intragastric balloon 1 is positioned in the chamber 44 in such a way that the exterior end of the valve 10 is pointing toward the proximal end 42 of the lead-in tube 41. The lead-in device 40 thus comprises an inflation tube 45, which is flexible at least in part and which is positioned inside the lead-in tube in such a way that it can move about during displacement. The distal end 56 of the inflation tube 45 is fitted with the inflation needle 25. The gastric balloon 1 and its lead-in device 40 are supplied to the physician in a ready-to-use state, as illustrated in FIG. 5. It is to be noted that in this state, the inflation tube 45 needle 25 is inserted into the reception channel 17 of the valve 10 without piercing the septum 19, and thus the integrity of the septum 19 is not affected. Long storage periods for the lead-in device and the balloon in this form can therefore be considered, without there being any risk of alteration to the isolation and impermeability properties of the valve 10.

Shortly prior to fitting the balloon, the operator will push the tube 45 inside the tube 42 in such a way as to insert the needle 25 fully into the valve, it being understood that the end of the needle 25 is to remain confined within the inflation channel 18, as depicted in FIG. 3. Next, the operator passes the lead-in device through the opening of the patient's pharynx and oesophagus, until the distal end of the device is placed inside the patient's stomach.

In order to ensure the release of the balloon, the operator ensures limited displacement of the lead-in tube 41 and the inflation tube 45, either by holding the lead-in tube 41 stationary and by pushing the inflation tube 45, or by holding the inflation tube 45 stationary and pulling the lead-in tube 41 out of it.

In order to guarantee the proper flow of the operator's efforts, the outer diameter of the inflation tube can be chosen so that it is essentially equal to the sliding clearance close to the inner diameter of the lead-in tube 41. Lubrication can also be considered using edible oil or biocompatible silicone oil. In the same way, in order to allow the physician a firmer grip on the proximal sections of the lead-in tube 41 and the inflation tube 45, a certain portion of these tubes can be made inflexible. Thus, the proximal section of the inflation tube can be inflexible along an upper portion greater than the length corresponding to the course of motion of the inflation tube in the lead-in tube 42. In order to guarantee the complete release of the balloon 2 before its inflation, the length of the inflation tube 45, excluding the needle 25, L₄₅ must be greater than or equal to the length L₄₁, outside the entire lead-in tube 41. The length L₄₅ can range between 50 cm and 100 cm and can for example be in the order of 57 cm. It must also be noted that the configuration adopted for the inflating valve and particularly for its elongate core, exerts expulsion force on the valve, so that with the needle inserted, the pouch of the intragastric balloon does not risk perforation.

After the intragastric balloon has been released, it can be filled up by means of the inflation tube, as has been previously described.

Once the filling has been completed, the operator removes the needle 25 from the inflating valve 10 by applying traction to the inflation tube 45 while holding the lead-in tube 41 steady. The inflated intragastric balloon 2 thus abuts the distal end 43 of the lead-in tube 41 which immobilises it during displacement and allows the needle 25 to be removed from the valve 10. The entire lead-in device 40 is finally removed from the patient's oesophagus.

According to the example illustrated, the chamber 44 is closed at the distal end 43 by elastically deformable flaps which limit abrasion of the oesophagus during insertion of the tube 45. However, in order to facilitate the placement of the folded intragastric balloon inside the receiving chamber 44 when the assembly is being fitted, the chamber 45 can also be sealed with a removable plug made of a biocompatible material which is able to dissolve in gastric juices and therefore be naturally eliminated. Such a plug which enables passage through the oesophagus can then be released without any risk when the balloon 1 is removed from the chamber 45 in the patient's stomach. This plug can be made with a starch-based food material, for example.

In all instances, for the purpose of limiting the abrasion of natural passageways, especially of the oesophagus during the fitting of the balloon 1, consideration can be given to the implementation of a over-tube with an appropriate inner diameter that enables easy passage of the lead-in device. The over-tube would therefore be fitted at the beginning of the procedure when the first inspection is conducted with an endoscope immediately prior to the fitting of the balloon 2. When the endoscope is removed, the over-tube remaining in place is used to give access to the lead-in device 40. Once the balloon has been filled and the lead-in device removed, the over-tube enables easy access for endoscopic inspection of the condition and position of the filled balloon inside the patient's stomach. In order to afford the patient respiratory comfort, the over-tube can have one or more lateral openings in the area intended to be at the point of connection of the larynx to the pharynx.

Similarly, in order to give the balloon 1 a surface area with as few protuberances as possible, a flange 49 can be used for smoothing the surface in the area where the valve 10 is attached to the balloon. This flange 49, represented as mixed lines in FIG. 1, can then be moulded or made an integral part of the core 11.

It is also possible to implement another insertion device such as is specifically illustrated in FIGS. 6 and 7. According to these forms of embodiment, the device comprises, as described in relation to FIG. 4, an endoscope cap 30 which has a body 30₁ defining the open chamber 31 at one distal end 32. The body 30₁ also defines an adaptor collar 33 on an endoscope. According to this example, the cap 30 additionally comprises a flexible elongate guide tube 50, one distal end 51 of which forms a single unit with the body 30₁, leading to the interior of the chamber 31. The guide tube 50 is therefore sufficiently long so that, with the cap 30 inside the patient's stomach, the proximal end of the guide tube 50 is located on the outside of the patient, having passed through the patient's mouth. The cap 30 additionally comprises an inflation tube 52 flexible at least in part. The inflation tube 52 is longer than the guide tube and is positioned on the inside of the guide tube so as to be movable during longitudinal displacement. The inflation tube 52 is fitted at one distal end 53 with the inflation needle 25.

The lead-in device comprises in addition a tubular sleeve 55 with openings at both the proximal 56 and distal 57 ends. The tubular sleeve 55 is therefore intended to be fitted onto the cap 30 at its distal end 32 as will be subsequently shown. The tubular sleeve 55 comprises in addition an elongate guide 56, the distal end 57 of which is attached to the sleeve 55. The guide 56 is therefore sufficiently long so that, with the sleeve 55 inside the patient's stomach, the proximal end (not represented) of the guide 56 is located on the outside of the patient, having passed through the patient's mouth. In order to make the guide 56 fairly rigid, this guide comprises a longitudinal core 58, positioned inside a sheath 57 made of flexible plastic material. The core 58 can be made from any suitable material such as for example metal in the form of a stranded cable or even a monofilament cable, of the type used for piano wire or similar, in stainless steel. The intragastric balloon 1 is then folded inside the sleeve 55, with the valve 10 located at the proximal end 56 of the sleeve 55.

The balloon and its lead-in device, composed as such, are then provided to the operator in the state illustrated in FIG. 6, with the free end of the needle 25 inserted into the lead-in chamber of the valve 10 without being embedded in the septum of the valve. The balloon 1 and its lead-in device can then be preserved in this state in blister packaging for long periods without any risk of modification to the properties of the valve 10.

When the operator is ready to install the balloon, he embeds the needle 25 fully into the valve 10, so that the septum is pierced. This insertion is performed manually, with the operator holding the balloon-sleeve assembly in one hand, and the distal end 53 of the inflation tube 52 in the other. Next, the operator fits the sleeve 55 onto the distal end 52 of the cap 30, so as to place the device in a configuration as illustrated in FIG. 7. The cap 30 can then be placed at the free end of an endoscope E, with the guide 56 and the guide tube 50 placed in such a way that they extend along the endoscope E.

In this state, the cap assembly 30, sleeve 55, balloon 1 and endoscope E are in a configuration essentially similar to the one described in relation to FIG. 4 and the installation of the balloon can then be performed, as previously described in relation to this same figure.

It should be noted, moreover, that the presence of the sleeve 55 and the guide 56 can be used to advantage to disconnect the sleeve from the cap, so as to enable the operator to use his endoscope to view the implantation configuration. This disconnection can be carried out at any time during the insertion, owing to the option provided by the guide 56 of applying pressure to the sleeve 55 independently of the movement of the cap 30. It should be noted that free displacement of the inflation tube 52 inside the guide tube 50 makes this relative movement possible without any risk of dislodgement of the needle 25. Additionally, the guide 56 immobilises the sleeve 55 in order to enable the free end of the cap to be reinserted into the sleeve, if the operator so desires.

FIGS. 8 and 9 represent a variant of embodiment, relative to an intragastric balloon 101 and an associated inflation needle 125.

The balloon 101 comprises a pouch 102, similar to the pouch 2 of the balloon 1 described previously, and an inflating valve 102 is impermeably sealed to the pouch 102. Similarly to the valve 10 of the balloon 1, the elongate core 111 of the valve 10 comprises, on the one hand, a channel 117 for receiving the needle 125, open at its external end 112 and, on the other hand, a coaxial inflation channel 118, open at its interior end 113, while a septum 119 separates the reception channel 117 from the inflation channel 118. Similarly, as in the case of the valve 10 of the balloon 1, the core 111 is encircled by a body 114 composed of a polymer material compatible with the constituent material of the pouch 102, this body 114 being itself encircled, on at least a part of the septum 119, by a prestressing ring 120.

This being said, the valve 110 is distinct from the valve 10 in two aspects, which can in addition be implemented independently of one other.

According to a first aspect of distinction, the reception channel 117 does not, like the channel 17, have a continuous transversal section along its entire length, but is equipped at one point along its longitudinal dimension, with a coaxial annular groove 117₁. In other words, this groove 177₁ is dug in the thickness of the core 111 from the surface delineating the channel 117, to form a hollow relief. In practical terms, the longitudinal span of the groove 177₁ is limited in relation to the total length of the channel 117, so that the presence of this groove does not affect the ability of the channel 117 to hold the needle 125 in place when the needle is inserted in the channel without necessarily being inserted in the septum 119, as shown in FIG. 8. In this respect, the considerations regarding configuration, particularly dimensional, mentioned in relation to the channels 17 and 18 and the needle 25 apply to the channels 117 and 118 and the needle 125 in order to ensure reliable, stable pre-positioning of the needle 125 in the reception channel 117, before the operator takes action with the needle to insert it into the septum 119 prior to installing the balloon 101 in a patient's stomach.

The importance of the groove 117₁ is shown in FIG. 9 which depicts the configuration of the balloon 101 and the needle 125 after the needle has been fully inserted into the septum 119. More specifically, in this configuration shown in FIG. 9, the proximal end 125₁ of the needle 125 is essentially located at the exterior end 112 of the valve 110 so that, as previously explained in detail, the free distal end 125₂ of the needle is located on the inside of the inflation channel 118. In this configuration, a raised annular flange 125₃, provided for in the current portion of the needle 125, near to its proximal end 125₁, snaps into, or more generally speaking, is mechanically gripped in the groove 117₁ of the reception channel 117. The interaction between this flange 125₃ and the groove 117₁ immobilises the needle 125 in relation to the valve 110, and in this way sets the configuration in FIG. 9. This positional interlocking between the valve 110 and the needle 125 guarantees that, once the operator has completely drawn the needle through the septum 119, this needle will not progressively withdrawn from the septum, particularly during subsequent manipulation of the assembly in order to install the balloon 101 in a patient's stomach.

Subsequent to this, once this balloon 101 has been installed in this way and is at least partly filled with liquid as previously explained, the needle 125 is disconnected from the valve 110, by applying enough traction pressure to remove the flange 125₃ from the groove 117₁, this pressure being easily applied by the operator.

In this connection, the proximal end 125₁ of the needle 125 is appropriately equipped with notches 125₄ shaped so as to be firmly anchored in the distal end of the inflation tube, like the tube 45 or the tube 52 described earlier, respectively, as compared to FIG. 5 and FIGS. 6 and 7. More specifically, in view of the shape of these notches 125₄, their stop action is significantly stronger in the direction indicated by the arrow F in FIG. 9, that is, in the direction corresponding to traction on the abovementioned inflation tube in order to disconnect the needle 125 from the valve 110.

In practical terms, reinforcement of the connection between the proximal end 125₁ and the distal end of the inflation tube, as a result of the notches, 125₄, is strengthened by a ring 127 moulded around the distal end of the inflation tube, in such a way as to apply centripetal force to this distal end.

According to the second aspect of distinction between the valve 10 of balloon 1 and the valve 110 of balloon 101, the septum 119 is not provided in the form of a ply of non-perforated material, before it is pierced by the needle 125, as in the case of the septum 19, but the septum 119 is traversed by a conduit 119₁ coaxially linking the reception channel 117 and the inflation channel 118. This traversing conduit 119₁ is extremely thin compared with channels 117 and 118, which explains why it is only shown as dotted lines in FIG. 8. In practical terms, the transversal thinness of this conduit 119₁ is such that, under the force of the body 114 and, above all, of the prestressing ring 120, this conduit 119₁ is impermeably sealed off, which explains why, in practical terms, it is almost invisible to the naked eye inside the valve 110. In contrast, this traversing conduit 119₁ is important in that it serves somewhat as a guidance pre-hole for the free distal end 125₂ of the needle 125 when the needle is being pressed down through the septum. In other words, the progression of the needle 125 is facilitated during its passage through the septum 119, as a result of a progressive separation from the conduit 119₁ under the action of the distal end of the needle, appropriated pointed, as shown in FIGS. 8 to 9. The material composing the septum 119 is therefore effectively shapeless, thereby limiting the risk of being damaged, from tearing, for example. This guarantees auto-sealing of the septum 119, once the needle 125 has been disconnected from the valve 110.

Naturally, the balloon 101 and the needle 125 are used as previously described for the balloon 1 and the needle 25, particularly in combination with the endoscope cap 30, the guide tubes 50 and inflation tubes 52 and/or the lead-in tubes and the inflation tubes 45.

As previously mentioned, examples of embodiments of inflation of the balloon 1 or 101 are given here following. By using 60 cm³ syringes and for an inflated balloon diameter of about 100 mm, it is recommended that the contents of two syringes of air be first injected, in order to free the balloon of its storage support devices (endoscope cap 30, chamber 44, sleeve 55 or similar), followed by the contents of:
either fourteen syringes of air in order to fill the balloon 100% with air,
or six syringes full of liquid and five syringes of air in order to fill the balloon 50% with air and 50% with liquid,
or nine syringes full of liquid in order to fill the balloon 100% with liquid.
   Similarly, still using 60 cm³ syringes and for an inflated balloon diameter of about 110 mm, it is recommended that the balloon 1 or 101 first be injected with the contents of two syringes of air in order to free the balloon of its storage support devices, followed by the contents of:
either sixteen syringes of air in order to fill the balloon 100% with air,
or seven syringes full of liquid and five syringes of air in order to fill the balloon 50% with air and 50% with liquid,
or ten syringes full of liquid in order to fill the balloon 100% with liquid.
   Note that the liquid mentioned above is, for example, a sterile saline solution or physiological saline solution.

1. An intragastric treatment assembly, comprising an intragastric balloon (1; 101) and an inflation needle (25; 125), such balloon comprising a flexible pouch (2; 102) sealed against air and food liquids or physiological liquids, as well as an inflating valve (10; 110) sealed onto the flexible pouch (2; 102) and comprising an elongate core (11; 111) made of elastomer, one end of which is accessible from the exterior of the balloon while the opposite end (13; 113) is located inside the balloon, which comprises:
a channel (17; 117) for receiving an inflation needle (25; 125), open at the exterior end (12; 112),
an inflation channel (18; 118) open at the interior end (13; 113) and coaxial with the reception channel (17; 117),
and a septum (19; 119) separating the reception channel (17; 117) from the inflation channel (18; 118),
wherein the reception channel (17; 117) and the inflation needle (25; 125) are adapted, by relative configuration, such that when the inflation needle is inserted into the reception channel without being inserted in the septum (19; 119), the inflation needle is held in place in the reception channel.

2. Assembly according to claim 1, wherein:
the inner diameter (d₁) of the reception channel (17; 117) is less than or equal to the outer diameter (Dₑ) of the inflation needle (25; 125),
the core (11; 111) of the valve has a length (L), measured between its exterior (12; 112) and interior ends (13; 113), which is greater than the usable length (Lu) of the inflation needle,
and the distance (D) between the exterior end of the core and the interior surface of the septum (19; 119) located in the inflation channel (18; 118), is shorter than the usable length (Lu) of the inflation needle.

3. Assembly according to claim 1, wherein the reception channel (117) and the needle (125) respectively are equipped with additional reliefs (117₁; 125₃) fitted so as to coordinate with each other and in this way immobilise the needle in the core (111) when the full usable length (Lu) of the needle is inserted into the reception channel, the septum (119) and one section at least of the inflation channel (118) at the same time.

4. Assembly according to claim 1, wherein the interior diameter (d₂) of the inflation channel (18; 118) is greater than the exterior diameter (Dₑ) of the inflation needle (25; 125).

5. Assembly according to claim 1, wherein the inflating valve (10; 110) comprises a body (14; 114) made of a polymer material, along the inside of which extends the elongate core (11; 111) and which is attached to the pouch (2; 102) of the balloon (1; 101).

6. Assembly according to claim 1, wherein the inflating valve (10; 110) comprises a prestressing ring (20; 120) encircling at least a part of the area of the core (11; 111) forming the septum (19; 119).

7. Assembly according to claim 6, wherein the prestressing ring (20; 120) is constructed from a rigid radio-opaque material, including metal.

8. Assembly according to claim 6, wherein the reception channel (117) and the inflation channel (118) are coaxially joined by a conduit (119₁) traversing the septum, which is adapted to guide the inflation needle (125) when it is being inserted into the septum (119) while, when the needle is not present in the septum, this conduit is impermeably sealed off under the action of the prestressing ring (120).

9. Assembly according to claim 1, wherein that the flexible pouch (2; 102) is constructed from one or more single-layer polyurethane sheets between 0.1 mm and 0.4 mm thick.

10. Assembly according to claim 1, wherein the flexible pouch (2; 102) is formed by the assembly of two disks (3, 4) of the same radius.

11. Assembly according to claim 1, further comprising an endoscope cap (30) comprising a chamber (31) open at a distal opening (32) and a tubular collar (33), to be fitted onto an endoscope, leading into the chamber (31) opposite the distal opening (32), and in that the balloon (1) is adapted to be folded in a deflated state inside the chamber (31) of the endoscope cap.

12. Assembly according to claim 1, further comprising:
an endoscope cap (30) comprising:
a body (30₁) defining a chamber (31) open at one distal end (32) and a tubular collar (33), to be fitted onto an endoscope, leading into the chamber (31) opposite the distal opening (32),
a guide tube (50), flexible and elongate, one distal end of which forms a single piece with the body and which leads to the interior of the chamber, and
an inflation tube (52), elongate and flexible in part at least, which is longer than the guide tube, which is positioned inside the guide tube so as to be movable during longitudinal displacement and which is fitted, at the distal end, with the inflation needle (25; 125),
and a tubular sleeve (55) which is open at the proximal (56) and distal (57) ends, which is designed to be fitted by its proximal end (56) to the distal end (32) of the endoscope cap (30) and which forms a single unit with an elongate guide (56) attached to the sleeve by one distal end,
and in that the balloon (1; 101) is adapted to be folded in a deflated state inside the sleeve, the inflating valve (10; 110) facing toward the distal end of the cap.

13. Assembly according to claim 12, wherein the guide (56) includes a metallic core (58).

14. Assembly according to claim 12, wherein the lead-in tube (41) has a length (L₄₁) ranging between 47 cm and 70 cm measured between the distal (43) and proximal ends (42) and in that the inflation tube (45) has a length (L₄₅) ranging between 50 cm and 100 cm.

15. Assembly according to-claim 1, further comprising a lead-in device (40) to be passed through the oesophagus, comprising:
a lead-in tube (41), elongate and flexible at least in part, which comprises a proximal end (42) and a distal end (43), which is sufficiently long so that, when the distal end (43) is located in a patient's stomach, the proximal end (42) is located outside of the patient, and which is equipped at the distal end with a tubular chamber (44) for receiving the balloon,
an inflation tube (45), elongate and flexible at least in part, which has a length (L45) greater than the length (L41) of the lead-in tube (41), which is positioned inside the lead-in tube (41) so as to be movable during longitudinal displacement and which is fitted, at the distal end, with the inflation needle (25; 125),
and in that the balloon (1; 101) is adapted to be folded in a deflated state inside the reception chamber (44), the inflating valve (10; 110) opposite the distal end (43) of the lead-in tube (41).

16. Assembly according to claim 15, wherein the lead-in tube (41) has a length (L₄₁) ranging between 47 cm and 70 cm measured between the distal (43) and proximal ends (42) and in that the inflation tube (45) has a length (L₄₅) ranging between 50 cm and 100 cm.

17. Assembly according to claim 14 wherein the inflation needle (125) is equipped at its proximal end (1250, with at least one notch (125₄) adapted to coordinate with the distal end of the inflation tube (45; 52) so as to firmly connect the needle to the inflation tube when traction is applied to the inflation tube to disconnect the needle from the valve (110).

18. Method for treating obesity of a patient comprising the following steps:
inserting an intragastric balloon in the patient's stomach,
filling the balloon within the patient's stomach with gas and liquid.

19. Method for treating obesity of a patient according to claim 18 wherein the balloon is filled half with gas and half with liquid.

20. Method for treating obesity of a patient according to claim 18 wherein the balloon is filled with a volume of liquid representing between 5% and 20% of the nominal volume of the balloon.
